(19) United States
(12) Patent Application Publication
(10) Pub. No.: US 2019/0099058 A1
(43) Pub. Date: Apr. 4, 2019

(54) MEDICAL OBSERVATION DEVICE AND MEDICAL OBSERVATION SYSTEM

(71) Applicant: Sony Olympus Medical Solutions Inc., Tokyo (JP)

(72) Inventor: Takayuki TAKEDA, Kanagawa (JP)

(73) Assignee: Sony Olympus Medical Solutions Inc., Tokyo (JP)

(21) Appl. No.: 16/143,815

(22) Filed: Sep. 27, 2018

(30) Foreign Application Priority Data
Oct. 3, 2017 (JP) 2017-193389

(51) Int. Cl. A61B 1/00; A61B 1/05; G02B 21/36; H04N 19/60; A61B 90/00

(52) U.S. Cl. CPC A61B 1/00009; A61B 1/051; G02B 21/24; H04N 19/60; A61B 90/361; G02B 21/365

(57) ABSTRACT

Provided is a medical observation device including: an encoding processing unit configured to compression-encode an image signal indicating a medical captured image by predetermined unit that is smaller than the medical captured image, the medical captured image being obtained by capturing an observation target by an imaging device; and a transmission unit configured to transmit the image signal compression-encoded by the predetermined unit.

### CROSS REFERENCE TO RELATED APPLICATIONS

This application claims the benefit of Japanese Priority Patent Application JP 2017-193389 filed Oct. 3, 2017, the entire contents of which are incorporated herein by reference.

### BACKGROUND

The present disclosure relates to a medical observation device and a medical observation system.

In recent years, medical observation devices which enlarge observation targets such as lesions for observation, for example, in order to support microsurgery like neurosurgical operations and to perform endoscopic surgery are used in the medical field. As medical observation devices, for example, medical observation devices with optical microscopes and medical observation devices with imaging devices functioning as electronic imaging-type microscopes are exemplified. Such a medical observation device with an optical microscope will be referred to as an “optical medical observation device” below. In addition, such a medical observation device with an imaging device will be referred to as an “electronic imaging-type medical observation device” or may be referred to simply as a “medical observation device” below. In addition, an image obtained by capturing an observation target using an imaging device included in a medical observation device will be referred to as a “medical captured image” below.

Electronic imaging-type medical observation devices are designed to obtain image quality equal to or higher than that of optical medical observation devices accompanied by high image quality of imaging devices and high resolution of display devices on which captured images are displayed. In addition, it is not necessary for users who use such electronic imaging-type medical observation devices (e.g., medical staff including operators, assistants of operators, etc.) to look into eyepieces of optical microscopes as in cases in which they use optical medical observation devices, and thus the users can move positions of imaging devices more freely. Thus, using electronic imaging-type medical observation devices is advantageous in that microsurgery and the like can be supported more flexibly, and thus use of electronic imaging-type medical observation devices has been progressing in the medical field.

In light of this, technologies relating to an endoscope system having an endoscope that compresses an image signal obtained from imaging and transmits the signal in a wireless manner have been developed. As a technology for an endoscope to perform a compression process at a compression ratio based on a determination result of a procedure, for example, the technology disclosed in WO 2016/052175 described below is exemplified.

### SUMMARY

An operator using an electronic imaging-type medical observation device or the like, for example, performs medical practice while viewing a display screen on which a medical captured image is displayed. Thus, assuming that an image signal indicating a medical captured image is compression-encoded and the compression-encoded image signal is transmitted in wired communication or wireless communication, further reducing influence of an error caused by the compression-encoding is considered to be necessary.

Here, the endoscope for which the technology disclosed in WO 2016/052175 is used performs a compression process at a compression ratio based on a determination result of a procedure and transmits a compressed image signal in a wireless manner. However, in the technology disclosed in WO 2016/052175, further reducing influence of an error caused by the compression-encoding is not taken into account.

The present disclosure proposes a novel and improved medical observation device and medical observation system that can transmit an image signal indicating a compression-encoded medical captured image while further reducing influence of an error caused by the compression-encoding.

According to an embodiment of the present disclosure, there is provided a medical observation device including: an encoding processing unit configured to compression-encode an image signal indicating a medical captured image by predetermined unit that is smaller than the medical captured image, the medical captured image being obtained by capturing an observation target by an imaging device; and a transmission unit configured to transmit the image signal compression-encoded by the predetermined unit.

In addition according to an embodiment of the present disclosure, there is provided a medical observation system including: a transmission side medical observation device including an encoding processing unit configured to compression-encode an image signal indicating a medical captured image by predetermined unit that is smaller than the medical captured image, the medical captured image being obtained by capturing an observation target by an imaging device, and a transmission unit configured to transmit the image signal compression-encoded by the predetermined unit; and a reception side medical observation device including a reception unit configured to receive the compression-encoded image signal transmitted from the transmission side medical observation device, and a signal processing unit configured to process the received compression-encoded image signal.

According to embodiments of the present disclosure, it is possible to transmit an image signal indicating a compression-encoded medical captured image while further reducing influence of an error caused by the compression-encoding.

Note that the effects described above are not necessarily limitative. With or in the place of the above effects, there may be achieved any one of the effects described in this specification or other effects that may be grasped from this specification.

### BRIEF DESCRIPTION OF THE DRAWINGS

FIG. 1 is an explanatory diagram illustrating a first example of a configuration of a medical observation system according to an embodiment of the present disclosure;

FIG. 2 is an explanatory diagram illustrating a second example of the configuration of the medical observation system according to an embodiment of the present disclosure

FIG. 3 shows explanatory diagrams for describing an example of a configuration of an imaging device included in the medical observation device illustrated in FIG. 2;

FIG. 4 is an explanatory diagram illustrating an example of transmission of an image signal indicating a medical captured image in a case in which the image signal is not compression-encoded;

FIG. 5 is an explanatory diagram illustrating an example of transmission of an image signal indicating a medical captured image in a case in which the image signal is compression-encoded;

FIG. 6 is an explanatory diagram for describing an example of an error caused by compression-encoding;

FIG. 7 is an explanatory diagram for describing an example of an error caused by compression-encoding in a case in which a compression-encoding method according to an embodiment of the present disclosure is applied; and

FIG. 8 is an explanatory diagram for describing an example of an effect exhibited by using the medical observation system according to an embodiment of the present disclosure.

### DETAILED DESCRIPTION OF THE EMBODIMENT(S)

Hereinafter, (a) preferred embodiment(s) of the present disclosure will be described in detail with reference to the appended drawings. Note that, in this specification and the appended drawings, structural elements that have substantially the same function and structure are denoted with the same reference numerals, and repeated explanation of these structural elements is omitted.

In addition, description will be provided below in the following order.
1. Medical observation system according to present embodiment and compression-encoding method according to present embodiment
2. Program according to present embodiment
   (Medical observation system according to present embodiment and compression-encoding method according to present embodiment)

An example of a medical observation system according to the present embodiment will be described, and then a compression-encoding method according to the present embodiment that can be applied to the medical observation system according to the present embodiment will be described below.

### [1] Configuration of Medical Observation System

### [1-1] Medical Observation System According to First Example

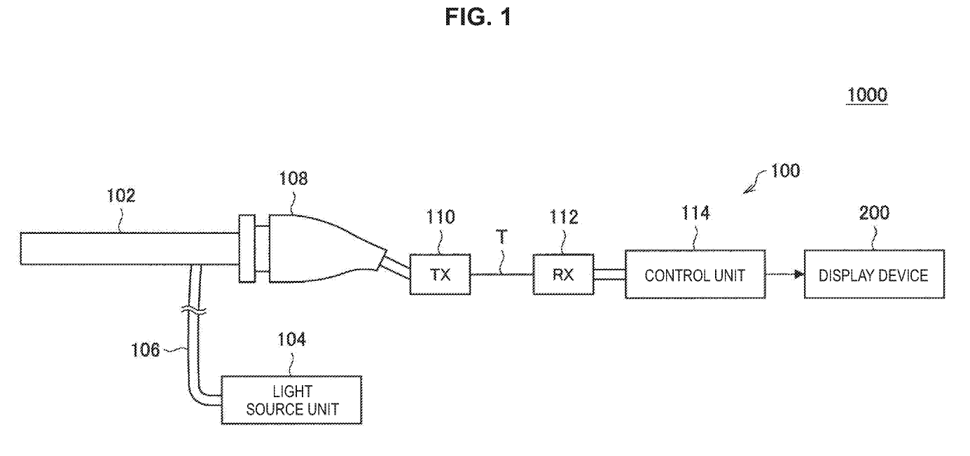

FIG. 1 is an explanatory diagram illustrating a first example of a configuration of a medical observation system 1000 according to the present embodiment, showing an example of a medical observation system having a medical observation device 100 that functions as an endoscope device that is an example of an electronic imaging-type medical observation device. The medical observation system 1000 illustrated in FIG. 1 has, for example, the medical observation device 100 and a display device 200.

Note that the medical observation system according to the first example is not limited to the example illustrated in FIG. 1.

The medical observation system according to the first example may further have a control device (not illustrated) that controls various operations of the medical observation device 100. The medical observation system 1000 illustrated in FIG. 11 is an example in which a control unit 114 (to be described later) has a function of a control device (not illustrated).

As the control device (not illustrated), for example, an arbitrary apparatus that can perform the process related to the compression-encoding method according to the present embodiment such as a “medical controller,” or a “computer such as a server” is exemplified. In addition, the control device (not illustrated) may be, for example, an integrated circuit (IC) that can be incorporated into the above-described apparatus.

In addition, the medical observation system according to the first example may have a plurality of medical observation devices 100 and display devices 200. In a case in which a plurality of medical observation devices 100 are provided, each of the medical observation devices 100 performs the process related to the compression-encoding method of the medical observation device 100 which will be described below. In addition, in the case in which the medical observation system according to the first example has a plurality of medical observation devices 100 and display devices 200, the medical observation devices 100 and the display devices 200 correspond to each other one to one, or the plurality of medical observation devices 100 may correspond to one display device 200. In the case in which the plurality of medical observation devices 100 correspond to one display device 200, for example, a switching operation is performed in the display device 200 to switch images captured by the medical observation devices 100 to be displayed on the display screen.

### [1-1-1] Display Device 200

The display device 200 is a display section of the medical observation system 1000, and corresponds to an external display device with respect to the medical observation device 100. The display device 200 displays various images, for example, medical captured images (moving images or a plurality of still images; the same applies below) captured by the medical observation device 100, images relating to a user interface, and the like. In addition, the display device 200 may be capable of performing 3D display. Display by the display device 200 is controlled by, for example, the medical observation device 100 or the control device (not illustrated).

The display device 200 of the medical observation system 1000 is installed in an arbitrary place at which the display device can be visually recognized by a person relating to surgery such as an operator within an operating room, for example, a wall surface, a ceiling, a floor of the operating room. As the display device 200, for example, a liquid crystal display, an organic electro-luminescence (EL) display, a cathode ray tube (CRT) display, or the like is exemplified.

Note that the display device 200 is not limited to the above-described example.

The display device 200 may be an arbitrary wearable device worn on the body of an operator or the like for use, for example, a head-mounted display, an eyewear-type device, or the like.

The display device 200 is driven by, for example, power supplied from an internal power supply included in the display device 200 such as a battery, power supplied from a connected external power supply, or the like.

### [1-1-2] Medical Observation Device 100

The medical observation device 100 constituting the medical observation system 1000 according to the first example is an endoscope device. In a case in which the medical observation device 100 illustrated in FIG. 1 is used during surgery, for example, an operator (an example of a user of the medical observation device 100) observes an operative site with reference to a medical captured image captured by the medical observation device 100 and displayed on the display screen of the display device 200, and performs various treatments such as a procedure on the operative site in accordance with a surgical technique.

The medical observation device 100 illustrated in FIG. 1 includes, for example, an insertion member 102, a light source unit 104, a light guide 106, a camera head 108, a transmitter 110, a receiver 112, and a control unit 114. The medical observation device 100 is driven by, for example, power supplied from an internal power supply included in the medical observation device 100 such as a battery, power supplied from a connected external power supply, or the like.

In FIG. 1, the transmitter 110 is denoted by “TX,” and the receiver 112 is denoted by “RX.” In addition, in FIG. 1, a communication path between the transmitter 110 and the receiver 112 is denoted by reference sign T. As the communication path T, a communication path for wired communication or a communication path for wireless communication is exemplified.

The insertion member 102 has an elongated shape and has an optical system that collects incident light therein. A tip of the insertion member 102 is inserted into, for example, a body cavity of a patient. A rear end of the insertion member 102 is connected to a tip of the camera head 108 to be detachable therefrom. In addition, the insertion member 102 is connected to the light source unit 104 via the light guide 106 and thus receives supply of light from the light source unit 104.

The insertion member 102 may have, for example, a material having no flexibility or of a material having flexibility. The medical observation device 100 can be called a rigid endoscope or a flexible endoscope depending on a material forming the insertion member 102.

The light source unit 104 is connected to the insertion member 102 via the light guide 106. The light source unit 104 supplies light to the insertion member 102 via the light guide 106. In addition, the light source unit 104 is connected by wire or wirelessly to the control unit 114, and light emitted from the light source unit 104 is controlled by the control unit 114.

Light supplied to the insertion member 102 is injected from the tip of the insertion member 102 and radiated to an observation target such as a tissue in a body cavity of a patient. In addition, light reflected from the observation target is collected by the optical system inside the insertion member 102.

The camera head 108 has a function of imaging an observation target. The camera head 108 is connected to the transmitter 110.

The camera head 108 includes an image sensor, captures the observation target by photoelectrically converting reflected light from the observation target collected by the insertion member 102, and transfers an image signal (a signal indicating a medical captured image) obtained from the imaging to the transmitter 110. As the image sensor of the camera head 108, for example, an image sensor using a plurality of image sensors such as complementary metal oxide semiconductors (CMOS), charge coupled devices (CCDs), and the like is exemplified.

In addition, the camera head 108 has, for example, a signal processing circuit, and the signal processing circuit compression-encodes an image signal indicating a medical captured image by performing the process of the compression-encoding method according to the present embodiment. That is, the signal processing circuit functions as an encoding processing unit that performs the process of the compression-encoding method according to the present embodiment in the medical observation device 100.

Note that the signal processing circuit may be provided in the transmitter 110. The process of the compression-encoding method according to the present embodiment will be described below.

In the medical observation device 100 functioning as an endoscope device, for example, the insertion member 102, the light source unit 104, and the camera head 108 play a role of an “imaging device that is inserted into the inside of the body of a patient and images the inside of the body.”

The transmitter 110 and the receiver 112 have a wired connection through an arbitrary signal line, for example, an optical fiber, a local area network (LAN) cable, or the like, on which an image signal indicating a medical captured image is transmitted and received in wired communication of an arbitrary communication method. In addition, the transmitter 110 and the receiver 112 are wirelessly connected through, for example, wireless communication of an arbitrary communication method such as optical communication, in which an image signal indicating a medical captured image is transmitted and received in the wireless communication.

The transmitter 110, for example, transmits an image signal compression-encoded in the process of the compression-encoding method according to the present embodiment, and the receiver 112 receives the compression-encoded image signal. Note that it is a matter of course that the transmitter 110 can transmit an image signal that has not undergone compression-encoding and the receiver 112 can receive an image signal that has not undergone compression-encoding.

That is, the transmitter 110 functions as a “transmission unit that transmits an image signal compression-encoded in the process of the compression-encoding method according to the present embodiment” in the medical observation device 100. In addition, the receiver 112 functions as a “reception unit that receives an image signal compression-encoded in the process of the compression-encoding method according to the present embodiment” in the medical observation device 100.

The transmitter 110 and the receiver 112 have hardware configurations supporting corresponding communication methods. In addition, the transmitter 110 may further have the signal processing circuit playing the role of performing the process of the compression-encoding method according to the present embodiment as described above.

The control unit 114 controls, for example, each of the light source unit 104 and the camera head 108.

In addition, the control unit 114 processes the image signal received by the receiver 112. To give an example of the signal processing of the control unit 114, the control unit 114 performs, for example, a decoding process of decoding the compression-encoded image signal. In addition, the control unit 114 may perform various processes that can be performed on the medical captured image, for example, adjustment of white balance, enlargement or reduction of an image using an electronic zoom function, inter-pixel correction, and the like.

In addition, the control unit 114 includes a communication device (not illustrated), and transmits an image signal received by the receiver 112 to the display device 200 in arbitrary wireless communication or arbitrary wired communication. The control unit 114 may transmit an image signal and a display control signal to the display device 200.

As the communication device (not illustrated) included in the control unit 114, for example, an IEEE 802.15.1 port and a transmission/reception circuit (wireless communication), an IEEE 802.11 port and a transmission/reception circuit (wireless communication), a communication antenna and an RF circuit (wireless communication), an optical communication device (wired communication or wireless communication), a LAN terminal and a transmission/reception circuit (wired communication), or the like are exemplified. The communication device (not illustrated) may be capable of communicating with one or two or more external devices using a plurality of communication methods.

Note that the control unit 114 may record the medical captured image.

As the control unit 114, for example, a camera control unit (CCU) is exemplified.

The medical observation device 100 functioning as an endoscope device has, for example, a hardware configuration illustrated with reference to FIG. 1. In the medical observation device 100 functioning as an endoscope device, for example, the insertion member 102, the light source unit 104, and the camera head 108 play the role of an imaging device, and the control unit 114 controls imaging of the imaging device.

In addition, in the medical observation device 100 illustrated in FIG. 1, for example, the image signal compression-encoded in the process of the compression-encoding method according to the present embodiment (or an image signal that has not undergone compression-encoding) is transmitted and received in communication between the transmitter 110 and the receiver 112. Thus, the medical observation device 100 illustrated in FIG. 1 can be understood as an example of, for example, a “medical observation system having a transmission-side medical observation device with members playing the role of an imaging device and the transmitter 110 and a reception-side medical observation device with the receiver 112 and the control unit 114.”

In addition, for example, switching (so-called replacement) of the transmitter 110 and the receiver 112 can be easier if each of the transmitter 110 and the receiver 112 is provided separately from other constituent elements such as the camera head 108, the control unit 114, and the like as illustrated in FIG. 1. Thus, by providing each of the transmitter 110 and the receiver 112 separately from other constituent elements, the medical observation device 100 can vary, for example, communication reliability, a communication distance, a transmission capacity (communication speed), an error correction method, a communication method, and the like more flexibly. That is, since the medical observation device 100 has the transmitter 110 and the receiver 112 that can each be switched and thus, for example, the version of the hardware relating to communication can be easily upgraded, the medical observation device 100 can respond to developments in communications more flexibly. In addition, the configuration in which each of the transmitter 110 and the receiver 112 can be switched is particularly effective for “transmission of an image signal in wireless communication in which communication reliability, a communication distance, a transmission capacity, and the like are objectives to be accomplished.” Note that it is a matter of course that the medical observation device 100 functioning as an endoscope can have, for example, a “configuration in which the camera head 108 includes the transmitter 110 and the control unit 114 includes the receiver 112.”

Note that the medical observation system 1000 according to the present embodiment is not limited to the configuration with the medical observation device 100 functioning as an endoscope device.

### [1-2] Medical Observation System According to Second Example

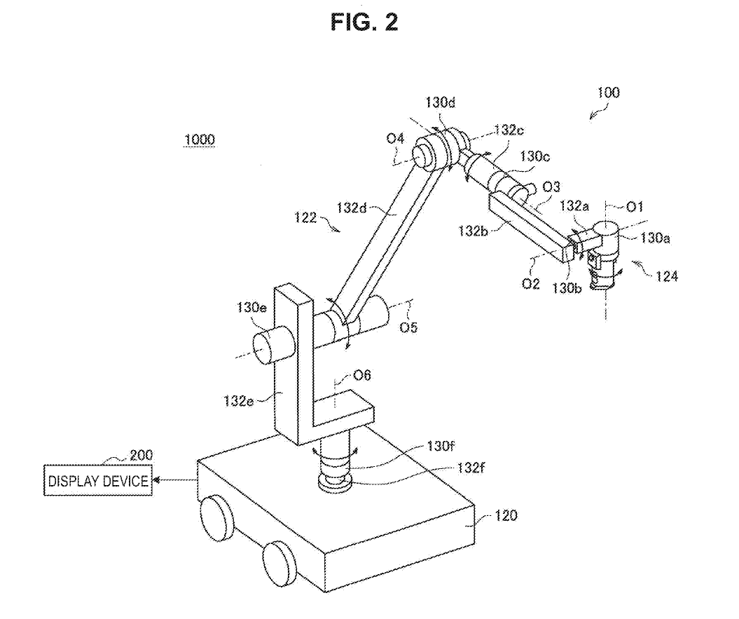

FIG. 2 is an explanatory diagram illustrating a second example of the configuration of the medical observation system 1000 according to the present embodiment, showing an example of the medical observation system having a medical observation device 100 functioning as an electronic imaging-type medical observation device according to another example. The medical observation system 1000 illustrated in FIG. 2 has, for example, the medical observation device 100 and a display device 200.

Note that the medical observation system according to the second example is not limited to the example illustrated in FIG. 2.

The medical observation system according to the second example may further have, for example, a control device (not illustrated) that controls various operations of the medical observation device 100, similarly to the medical observation system according to the first example.

In addition, the medical observation system according to the second example may have a plurality of medical observation devices 100 and a plurality of display devices 200, similarly to the medical observation system according to the first example.

### [1-2-1] Display Device 200

The display device 200 constituting the medical observation system according to the second example has a similar function and configuration to the display device 200 constituting the medical observation system according to the first example.

### [1-2-2] Medical Observation Device 100

The medical observation device 100 constituting the medical observation system 1000 according to the second example is an electronic imaging-type medical observation device according to another example. An example of a hardware configuration of the medical observation device 100 functioning as an electronic imaging-type medical observation device will be described with reference to FIG. 2.

The medical observation device 100 functioning as an electronic imaging-type medical observation device includes, for example, a base 120, an arm 122, and an imaging device 124.

In addition, although not illustrated in FIG. 2, the medical observation device 100 may also include, for example, one or two or more processors (not illustrated) constituted by an arithmetic circuit such as a micro-processing unit (MPU), a read only memory (ROM; not illustrated), a random access memory (RAM; not illustrated), and a recording medium (not illustrated), and a communication device (not illustrated). The medical observation device 100 is driven by, for example, power supplied from an internal power supply included in the medical observation device 100 such as a battery, power supplied from a connected external power supply, or the like.

The processors (not illustrated) function as a control unit (not illustrated) that controls the entire medical observation device 100. The ROM (not illustrated) stores control data such as programs and arithmetic parameters to be used by the processors (not illustrated). The RAM (not illustrated) temporarily stores programs executed by the processors (not illustrated) and the like.

The recording medium (not illustrated) functions as a storage unit. The recording medium (not illustrated) stores, for example, various kinds of data such as data relating to the compression-encoding method according to the present embodiment such as data indicating thresholds or data indicating compression ratios and various applications. Here, as the recording medium (not illustrated), for example, a magnetic recording medium such as a hard disk, a non-volatile memory such as a flash memory, or the like is exemplified. In addition, the recording medium (not illustrated) may be detachable from the medical observation device 100.

The communication device (not illustrated) is a communication section included in the medical observation device 100, and plays a role of performing wireless or wired communication with an external device such as the display device 200. Here, as the communication device (not illustrated), for example, an IEEE 802.15.1 port and a transmission/reception circuit, an IEEE 802.11 port and a transmission/reception circuit, a communication antenna and an RF circuit, an optical communication device, a LAN terminal and a transmission/reception circuit, or the like are exemplified. The communication device (not illustrated) may be capable of communicating with one or two or more external devices in a plurality of communication methods.

### [1-2-2-1] Base 120

The base 120 is the base of the medical observation device 100, and is connected to one end of the arm 122 to support the arm 122 and the imaging device 124.

In addition, the base 120 has, for example, casters, and the medical observation device 100 stands on the floor via the casters. By having the casters, the medical observation device 100 can be easily moved on the floor with the casters.

### [1-2-2-2] Arm 122

The arm 122 is constituted by a plurality of links connected to each other by joints.

In addition, the arm 122 supports the imaging device 124. The imaging device 124 supported by the arm 122 is three-dimensionally movable, and the arm 122 helps the imaging device 124 to maintain a position and a posture after movement.

More specifically, the arm 122 is constituted by, for example, a plurality of joints 130a, 130b, 130c, 130d, 130e, and 130f and a plurality of links 132a, 132b, 132c, 132d, 132e, and 132f that are connected by the joints 130a, 130b, 130c, 130d, 130e, and 130f to revolve. A rotatable range of each of the joints 130a, 130b, 130c, 130d, 130e, and 130f is arbitrarily set in the design stage, the manufacturing stage, or the like so that desired movement of the arm 122 is realized.

That is, in the medical observation device 100 illustrated in FIG. 2, six degrees of freedom with respect to movement of the imaging device 124 are realized by six rotation axes (a first axis O1, a second axis O2, a third axis O3, a fourth axis O4, a fifth axis O5, and a sixth axis O6) corresponding to the six joints 130a, 130b, 130c, 130d, 130e, and 130f constituting the arm 122. More specifically, in the medical observation device 100 illustrated in FIG. 2, movement of six degrees of freedom including three translational degrees of freedom and three rotational degrees of freedom is realized.

Each of the joints 130a, 130b, 130c, 130d, 130e, and 130f has an actuator (not illustrated), and each of the joints 130a, 130b, 130c, 130d, 130e, and 130f rotates at a corresponding rotational axis by driving of the actuator (not illustrated). Driving of the actuator (not illustrated) is controlled by, for example, a processor functioning as a control unit (not illustrated) or an external control device (not illustrated).

Since each of the joints 130a, 130b, 130c, 130d, 130e, and 130f rotates at a corresponding rotational axis by driving of the actuator (not illustrated), various kinds of operations of the arm 122, for example, stretching, shrinking (folding), and the like of the arm 122, are realized.

The joint 130a has a substantially cylindrical shape, and supports the imaging device 124 (an upper end portion of the imaging device 124 in FIG. 2) to be revolvable around a rotation axis (the first axis O1) parallel to a central axis of the imaging device 124 at a tip portion of the joints 130a (a lower end part thereof in FIG. 2). Here, the medical observation device 100 is configured such that the first axis O1 matches the optical axis of the imaging device 124. That is, by causing the imaging device 124 to revolve around the first axis O1 illustrated in FIG. 2, a medical captured image captured by the imaging device 124 becomes an image in which a line of sight is changed to rotate.

The link 132a is a substantially rod-shaped member, and fixedly supports the joint 130a. The link 132a extends, for example, in a direction orthogonal to the first axis O1 and is connected to the joint 130b.

The joint 130b has a substantially cylindrical shape and supports the link 132a to be revolvable around the rotation axis (the second axis O2) orthogonal to the first axis O1. In addition, the link 132b is fixedly connected to the joint 130b.

The link 132b is a substantially rod-shaped member and extends in a direction orthogonal to the second axis O2. In addition, the joint 130b and the joint 130c are respectively connected to the link 132b.

The joint 130c has a substantially cylindrical shape and supports the link 132b to be revolvable around the rotation axis (the third axis O3) orthogonal to the first axis O1 and the second axis O2. In addition, one end of the link 132c is fixedly connected to the joint 130c.

Here, by causing the tip side of the arm 122 (the side on which the imaging device 124 is provided) to revolve around the second axis O2 and the third axis O3, the imaging device 124 can be moved so that a position of the imaging device 124 is changed within a horizontal plane. That is, since rotation around the second axis O2 and the third axis O3 is controlled in the medical observation device 100, a line of sight of a medical captured image can be moved within a plane.

The link 132c has a member having one end in a substantially cylindrical shape and the other end in substantially a rod shape. The one end of the link 132c is fixedly connected to the joint 130c such that the central axis thereof and the central axis of the substantially cylindrical shape are the same. In addition, the other end of the link 132c is connected to the joint 130d.

The joint 130d has a substantially cylindrical shape and supports the link 132c to be revolvable around a rotational axis (the fourth axis O4) orthogonal to the third axis O3. The link 132d is fixedly connected to the joint 130d.

The link 132d is a substantially rod-shaped member and extends to be orthogonal to the fourth axis O4. One end of the link 132d is fixedly connected to the joints 130d to abut against a side face of the substantially cylindrical shape of the joint 130d. In addition, the other end of the link 132d (the end on the opposite side to the side on which the joint 130d is connected) is connected to the joint 130e.

The joint 130e has a substantially cylindrical shape and supports one end of the link 132d to be revolvable around the rotational axis (the fifth axis O5) parallel to the fourth axis O4. In addition, the joint 130e is connected to one end of the link 132e.

Here, the fourth axis O4 and the fifth axis O5 are rotational axis that can move the imaging device 124 in the vertical direction. By causing the tip side of the arm 122 (the side on which the imaging device 124 is provided) to revolve around the fourth axis O4 and the fifth axis O5, a position of the imaging device 124 in the vertical direction is changed. Thus, by causing the tip side of the arm 122 (the side on which the imaging device 124 is provided) to revolve around the fourth axis O4 and the fifth axis O5, a distance between the imaging device 124 and an observation target such as an operative site of a patient or the like can be changed.

The link 132e is a member constituted by a combination of a first member having a substantially L shape with one side extending in the vertical direction and the other side extending in the horizontal direction and a rod-shaped second member extending vertically downward from a portion of the first member extending in the horizontal direction. A portion of the first member of the link 132e extending in the vertical direction is fixedly connected to the joint 130e. In addition, the second member of the link 132e is connected to the joint 130f.

The joint 130f has a substantially cylindrical shape and supports the link 132e to be revolvable around a rotational axis (the sixth axis O6) parallel to the vertical direction. In addition, the joint 130f is fixedly connected to the link 132f.

The link 132f is a substantially rod-shaped member and extends in the vertical direction. One end of the link 132f is connected to the joint 130f. In addition, the other end of the link 132f (the end on the opposite side to the side on which the joints 130f is connected) is fixedly connected to the base 120.

Since the arm 122 has the above-described configuration, six degrees of freedom with respect to movement of the imaging device 124 are realized in the medical observation device 100.

Note that a configuration of the arm 122 is not limited to the above-described example.

For example, a brake that regulates rotation of each of the joints 130a, 130b, 130c, 130d, 130e, and 130f may be provided in each of the joints 130a, 130b, 130c, 130d, 130e, and 130f of the arm 122. As a brake according to the present embodiment, for example, an arbitrary type of brake such as a mechanically driven brake or an electrically driven electromagnetic brake is exemplified.

Driving of the brake is controlled by, for example, a processor that functions as a control unit (not illustrated) or an external control device (not illustrated). Since driving of the brake is controlled, an operation mode of the arm 122 is set in the medical observation device 100. As operation modes of the arm 122, for example, a fixed mode and a free mode are exemplified.

Here, the fixed mode according to the present embodiment is an operation mode in which, for example, a position and a posture of the imaging device 124 are fixed by a brake regulating rotation at each rotational axis provided in the arm 122. When the arm 122 is the fixed mode, an operation state of the medical observation device 100 is a fixed state in which a position and a posture of the imaging device 124 are fixed.

In addition, the free mode according to the present embodiment is an operation mode in which, when the brake is released, each rotational axis provided in the arm 122 is freely rotatable. In the free mode, for example, a position and a posture of the imaging device 124 can be adjusted through a direct operation by an operator. Here, a direct operation according to the present embodiment means, for example, an operation in which an operator grabs the imaging device 124 with his or her hand and moves the imaging device 124 in person.

### [1-2-2-3] Imaging Device 124

The imaging device 124 is supported by the arm 122 and captures an observation target, for example, an operative site of a patient, or the like. Imaging by the imaging device 124 is controlled by, for example, a processor that functions as a control unit (not illustrated) or an external control device (not illustrated).

The imaging device 124 has a configuration corresponding to, for example, an electronic imaging-type microscope.

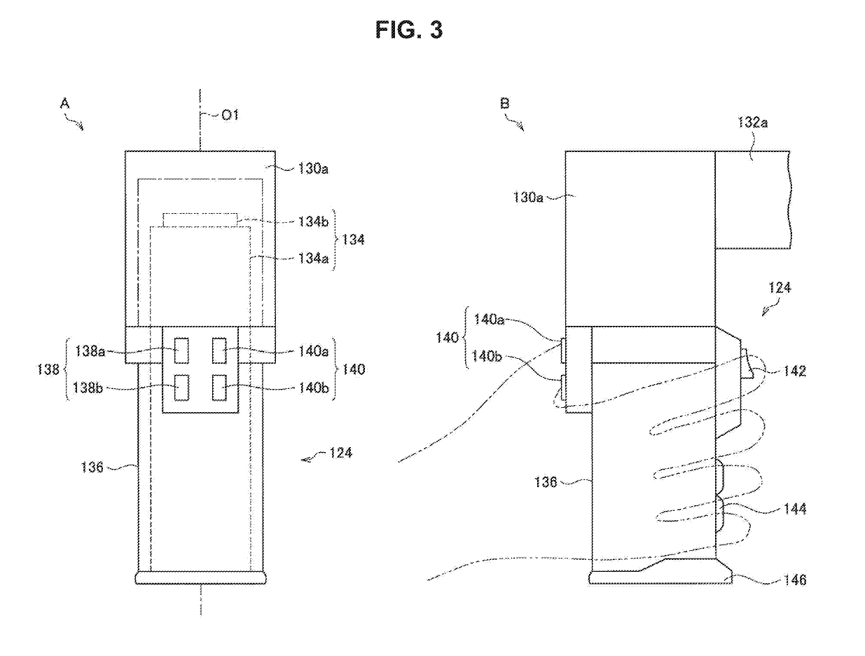

FIG. 3 shows explanatory diagrams for describing an example of a configuration of the imaging device 124 included in the medical observation device 100 illustrated in FIG. 2.

The imaging device 124 has, for example, an imaging member 134 and a tubular member 136 having a substantially cylindrical shape, and the imaging member 134 is provided in the tubular member 136.

Cover glass (not illustrated) for protecting the imaging member 134, for example, is provided on an opening surface of a lower end (an end on a lower side in FIG. 3) of the tubular member 136.

In addition, a light source (not illustrated) is provided, for example, inside of the tubular member 136, and during imaging, illumination light from the light source is radiated to a subject through the cover glass. Since reflected light (observation light) from the subject irradiated with the illumination light is incident on the imaging member 134 through the cover glass (not illustrated), an image signal (an image signal indicating a captured image) representing the subject is obtained by the imaging member 134.

As the imaging member 134, a configuration used in any of various known electronic imaging-type microscope unit can be applied.

To give an example, the imaging member 134 is constituted by, for example, an optical system 134a and an image sensor 134b including an image sensor that captures image of an observation target using light that has passed through the optical system 134a. The optical system 134a includes optical elements, for example, one or two or more lenses such as an objective lens, a zoom lens, and a focus lens, a mirror, and the like. As the image sensor 134b, for example, an image sensor using a plurality of image sensors such as complementary metal oxide semiconductors (CMOS), charge coupled devices (CCDs), and the like is exemplified.

The imaging member 134 may have a pair of image sensors, that is, may function as a so-called stereo camera. The imaging member 134 may have one or two or more functions included in a general electronic imaging type microscope unit, such as a zoom function (one or both of an optical zoom function and an electronic zoom function), a focus function such as auto focus (AF), and the like.

In addition, the imaging member 134 may be capable of perform imaging at so-called high resolution of, for example, 4K, 8K, or the like. When the imaging member 134 can perform imaging at high resolution, it is possible to display an image on the display device 200 having a large display screen of, for example, 50 inches or greater while predetermined resolution (e.g., full HD image quality, etc.) is secured, and thus visibility of an operator viewing the display screen is improved. In addition, when the imaging member 134 can perform imaging at high resolution, even if a captured image is enlarged using the electronic zoom function and displayed on the display screen of the display device 200, predetermined resolution can be secured. Furthermore, in a case in which predetermined resolution is secured by using the electronic zoom function, performance of the optical zoom function of the imaging device 124 can be suppressed, and thus the optical system of the imaging device 124 can be made simpler and thus the imaging device 124 can be further miniaturized.

The imaging device 124 has, for example, various operation devices for controlling operations of the imaging device 124. In FIG. 3, for example, a zoom switch 138, a focus switch 140, and an operation mode change switch 142 are provided in the imaging device 124. Note that it is a matter of course that a position at which the zoom switch 138, the focus switch 140, and the operation mode change switch 142 are provided and shapes thereof are not limited to the example illustrated in FIG. 3.

The zoom switch 138 and the focus switch 140 are an example of an operation device for adjusting imaging conditions of the imaging device 124.

The zoom switch 138 is constituted by, for example, a zoom-in switch 124a for increasing zoom magnifications (enlargement magnifications) and a zoom-out switch 124b for decreasing zoom magnifications. A zoom magnification is adjusted by performing an operation on the zoom switch 138, and thereby zoom is adjusted.

The focus switch 140 is constituted by, for example, a distant view focus switch 140a for lengthening a focal distance to an observation target (subject) and a near-view focus switch 140b for shortening a focal distance to an observation target. By adjusting a focal distance by performing an operation on the focus switch 140, focus is adjusted. Lengthening a focal distance to an observation target may be called “focus out,” and shortening a focal distance to an observation target may be called “focus in.”

The operation mode change switch 142 is an example of an operation device of the imaging device 124 for changing an operation mode of the arm 122. When an operation is performed on the operation mode change switch 142, the operation mode of the arm 122 is changed. As the operation mode of the arm 122, for example, there are the fixed mode and the free mode as described above.

As an example of an operation with respect to the operation mode change switch 142, an operation of pressing the operation mode change switch 142 is exemplified. For example, while an operator presses the operation mode change switch 142, the operation mode of the arm 122 shifts to the free mode, and when the operator does not press the operation mode change switch 142, the operation mode of the arm 122 shifts to the fixed mode.

In addition, in the imaging device 124, a non-slip member 144 and a projecting member 146, for example, are provided to improve operability, convenience, and the like during operations by an operator who performs an operation with respect to the various operation devices.

The non-slip member 144 is a member provided to prevent an operating body from slipping when, for example, an operator performs an operation on the tubular member 136 using an operating body such as his or her hand. The non-slip member 144 has, for example, a material having a high friction factor, and thus has a structure which makes it difficult for an operating body to slip due to unevenness.

The projecting member 146 is a member provided to prevent an operating body from blocking a visual field of the optical system 134a when an operator operates the tubular member 136 with the operating body such as his or her hand or to prevent the cover glass (not illustrated) from becoming dirty due to contact of the cover glass with an operating body when performing an operation with the operating body.

Note that it is a matter of course that a position at which each of the non-slip member 144 and the projecting member 146 is provided and a shape thereof are not limited to the example illustrated in FIG. 3. In addition, in the imaging device 124, one or both of the non-slip member 144 and the projecting member 146 may not be provided.

An image signal (image data) generated through imaging by the imaging device 124 is transmitted or received by, for example, a transmitter (not illustrated) and a receiver (not illustrated) having similar functions and configurations as the transmitter 110 and the receiver 112 illustrated in FIG. 1 in wired communication or wireless communication. In the medical observation device illustrated in FIG. 2, a signal processing circuit functioning as an encoding processing unit may be included in the imaging device 124 or the transmitter described above.

In addition, the image signal generated through imaging by the imaging device 124 is processed by, for example, the processor functioning as a control unit (not illustrated). As image processing according to the present embodiment, for example, one or two or more types of processing among various kinds of processing such as gamma correction, adjustment of white balance, enlargement or reduction of an image using the electronic zoom function, inter-pixel correction, and the like are exemplified.

In a case in which the image signal generated through imaging by the imaging device 124 is processed by the processor functioning as a control unit (not illustrated), the medical observation device 100 illustrated in FIG. 2 can be understood as, for example, an example of a “medical observation system having a transmission side medical observation device with the imaging device 124 and the transmitter and a reception side medical observation device with the receiver and the processor.”

Note that, in a case in which the medical observation system according to the second example has a control device (not illustrated) that controls various operations of the medical observation device 100, the image processing according to the present embodiment may be performed by the control device (not illustrated). In this case, an image signal generated through imaging by the imaging device 124 is transmitted by, for example, the transmitter (not illustrated) having a similar function and configuration as the transmitter 110 illustrated in FIG. 1 in wired or wireless communication, and processed by the control device (not illustrated). In addition, in that case, the medical observation device 100 plays the role of the transmission side medical observation device, and the control device (not illustrated) plays the role of the reception side medical observation device.

The medical observation device 100 transmits, for example, a display control signal and an image signal that has undergone the above-described image processing to the display device 200.

When a display control signal and an image signal are transmitted to the display device 200, the display screen of the display device 200 displays a medical captured image obtained by capturing an observation target (e.g., a captured image in which an operative site is captured) enlarged or reduced to a desired magnification using one or both of the optical zoom function and the electronic zoom function.

The medical observation device 100 that functions as the electronic imaging-type medical observation device according to the other example has, for example, the hardware configuration illustrated with reference to FIGS. 2 and 3.

Note that a hardware configuration of the medical observation device that functions as the electronic imaging-type medical observation device according to the other example is not limited to the configuration illustrated with reference to FIGS. 2 and 3.

For example, the medical observation device according to the present embodiment may have the arm 122 that is directly installed on a ceiling, a wall surface, or the like of an operating room or the like, without having the base 120. For example, in a case in which the arm 122 is installed on a ceiling, the arm 122 of the medical observation device according to the present embodiment is hung from the ceiling.

In addition, although the example in which the arm 122 realizes six degrees of freedom with respect to driving of the imaging device 124 is illustrated in FIG. 2, a configuration of the arm 122 is not limited to the configuration in which driving of the imaging device 124 has six degrees of freedom. For example, the arm 122 may appropriately move the imaging device 124 in accordance with an application, and the number and disposition of the joints and links, directions of driving axes of the joints, and the like can be appropriately set so that the arm 122 has a desired degree of freedom. To give an example, the medical observation device according to the present embodiment may have a simpler configuration of controlling an X axis and a Y axis, like an ophthalmology microscope.

In addition, although the example in which various operation devices for controlling operations of the imaging device 124 are provided in the imaging device 124 is illustrated in FIGS. 2 and 3, some or all of the operation devices illustrated in FIGS. 2 and 3 may not be provided in the imaging device 124. To give an example, various operation devices for controlling operations of the imaging device 124 may be provided in a part other than the imaging device 124 constituting the medical observation device according to the present embodiment. In addition, to give another example, various operation device for controlling operations of the imaging device 124 may be external operation devices such as a foot switch and a remote controller.

As the medical observation device 100 constituting the medical observation system 1000 according to the present embodiment, for example, a medical observation device that functions as the endoscope device illustrated in FIG. 1, a medical observation device that functions as the electronic imaging-type medical observation device according to the other example illustrated in FIG. 2, or the like is exemplified.

### [2] Compression Encoding Method According to the Present Embodiment

### [2-1] Overview of Compression-Encoding Method According to the Present Embodiment

In electronic imaging-type medical observation devices of recent years, amounts of image signals indicating medical captured images have tended to increase resulting from, for example, high resolution of imaging devices, high frame rates, stereoscopy, mounting of additional devices for observing special light incident on the imaging devices, and the like. An image signal indicating a medical captured image may be simply referred to as an “image signal” below.

When an amount of an image signal increases as described above, power consumption of the medical observation device increases accordingly. In addition, since an amount of heat generation increases due to the increase of power consumption, the sizes of members (e.g., the size of the camera head constituting the endoscope device) constituting the medical observation device should be increased in order to deal with heat. Therefore, an increase in an amount of an image signal is disadvantageous for miniaturizing the medical observation device.

Here, as a method of reducing an amount of an image signal, compression-encoding of the image signal is considered.

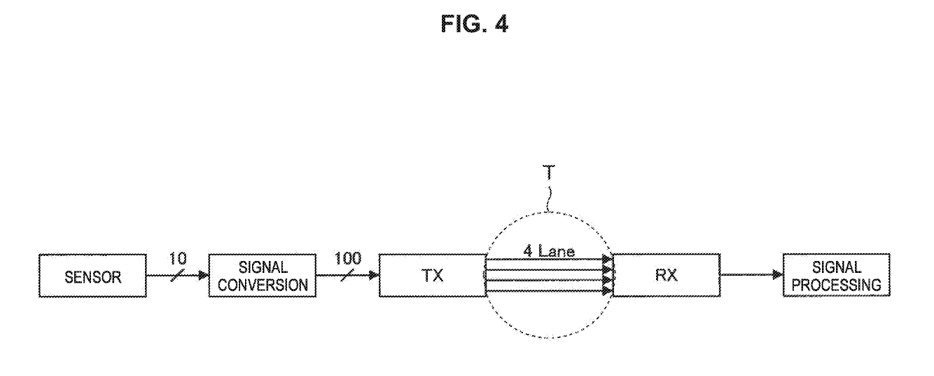

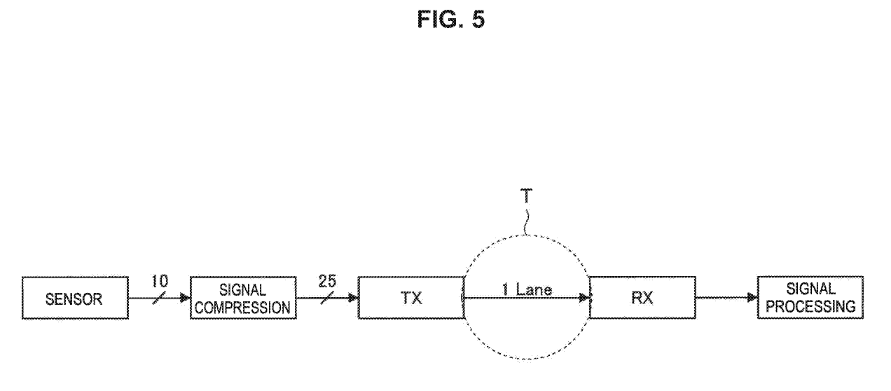

FIG. 4 is an explanatory diagram illustrating an example of transmission of an image signal indicating a medical captured image in a case in which the image signal is not compression-encoded. In addition, FIG. 5 is an explanatory diagram illustrating an example of transmission of an image signal indicating a medical captured image in a case in which the image signal is compression-encoded. In FIGS. 4 and 5, image sensors included in the imaging devices are denoted by “sensor.” In addition, in FIGS. 4 and 5, transmitters are denoted by “TX,” and receivers are denoted by “RX.”

In the case in which an image signal indicating a medical captured image is not compression-encoded, for example, the image signal is transmitted from the transmitter to the receiver on a 4-lane communication path T as illustrated in FIG. 4.

On the other hand, in a case in which an image signal indicating a medical captured image is compression-encoded at a compression ratio of 1/4, the compression-encoded image signal is transmitted from the transmitter to the receiver on one lane of the communication path T, for example, as illustrated in FIG. 5. In addition, in a case in which an image signal is compression-encoded at a compression ratio of 1/8, the image signal indicating a medical captured image functioning as a stereo image can be transmitted on one lane of the communication path T.

By compression-encoding the image signal as illustrated in FIG. 5, for example, the amount of the image signal can be reduced in accordance with the compression ratio and degradation of the quality of the decoded medical captured image can be prevented.

However, when it is assumed that the image signal indicating a medical captured image is compression-encoded and the compression-encoded image signal is transmitted in wired or wireless communication as described above, the necessity of further reducing influence of an error caused by the compression-encoding is considered.

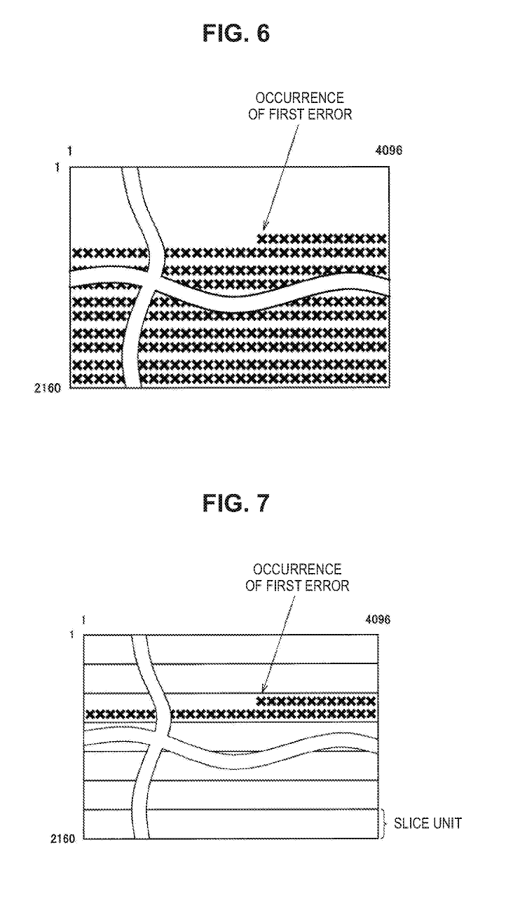

FIG. 6 is an explanatory diagram for describing an example of an error caused by compression-encoding. FIG. 6 conceptually illustrates an error occurring in a case in which an entire medical captured image with 4K resolution (4096×2160 pixels) of a certain frame (an example of an entire frame image) is compression-encoded.

In the case in which the entire medical captured image is compression-encoded as illustrated in, for example, FIG. 6, when an error occurs in the course of compression-encoding, it is not possible to decode pixels from the pixel at which the error occurs to the final pixel of the frame to be processed after the occurrence of the error. That is, in the case in which the entire medical captured image is compression-encoded, the influence of the error occurring in the course of the compression-encoding is propagated to all pixels processed after the occurrence of the error.

Thus, in the medical observation system 1000, the image signal indicating a medical captured image is compression-encoded by each predetermined unit that is smaller than the medical captured image.

As the predetermined unit according to the present embodiment, for example, the unit of a plurality of lines of the medical captured image, such as every 16 lines, is exemplified. Here, the predetermined unit may be a preset fixed unit or a variable unit that can be changed on the basis of an operation of a user using the medical observation system 1000, an operation state of a predetermined medical apparatus, or the like.

Note that the predetermined unit according to the present embodiment is not limited to a unit of a plurality of lines of a medical captured image. The predetermined unit according to the present embodiment may be, for example, a block unit that includes a plurality of pixels and is smaller than the entire medical captured image. Hereinbelow, a case in which the predetermined unit according to the present embodiment is a unit of a plurality of lines of a medical captured image will be exemplified. In addition, hereinbelow, the predetermined unit defined as a unit of a plurality of lines of a medical captured image may be indicated as a “slice unit.”

FIG. 7 is an explanatory diagram for describing an example of an error caused by compression-encoding in a case in which the compression-encoding method according to the present embodiment is applied. FIG. 7 conceptually illustrates an error occurring in a case in which an entire medical captured image of 4K resolution of a certain frame is compression-encoded, as in FIG. 6.

In a case in which a medical captured image is compression-encoded by a slice unit (an example of a predetermined unit), for example, as illustrated in FIG. 7, even if an error occurs in the course of the compression-encoding, propagation of the error stops at a slice unit in which the error has occurred, and the influence thereof does not spread to slice units processed thereafter.

Therefore, by using the compression-encoding method according to the present embodiment, the amount of the image signal can be reduced while further reducing the influence of the error caused by the compression-encoding.

In addition, in a case in which the medical captured image is compression-encoded by a smaller predetermined unit, for example, like the slice unit as illustrated in FIG. 7, the time taken to perform compression-encoding by the predetermined unit becomes shorter than in the case in which the entire medical captured image is compression-encoded as illustrated in FIG. 6. Therefore, in the case in which the compression-encoding method according to the present embodiment is used, the compression-encoded image signal can be transmitted with lower latency than in the case in which the entire medical captured image is compression-encoded as illustrated in FIG. 6. In addition, such transmission of an image signal with low latency is beneficial when medical staff performing medical practice while viewing a decoded medical captured image are taken into account.

### [2-2] Process of Medical Observation Device 100 to Which Compression-Encoding Method According to Present Embodiment is Applied

Next, an example of a process of a medical observation device 100 to which the above-described compression-encoding method according to the present embodiment is applied will be described for each functional block. The example of the process of the medical observation device 100 will be described below exemplifying a case in which the medical observation device 100 is the medical observation device 100 constituting the medical observation system 1000 according to the first example illustrated in FIG. 1.

The medical observation device 100 has, for example, an encoding processing unit and a transmission unit. The medical observation device 100 having the encoding processing unit and the transmission unit functions as a transmission side medical observation device.

As the encoding processing unit, for example, a signal processing circuit provided in the medical observation device 100 is exemplified. The signal processing circuit of the medical observation device 100 illustrated in FIG. 1 is provided in the camera head 108 or the transmitter 110, for example, as described above.

As the transmission unit, the transmitter 110 is exemplified.

In addition, the medical observation device 100 may further have, for example, a reception unit and a signal processing unit. The medical observation device 100 having the reception unit and the signal processing unit functions as a reception side medical observation device.

As the reception unit, the receiver 112 is exemplified.

As the signal processing unit, for example, the control unit 114 is exemplified.

Each of the encoding processing unit, the transmission unit, the reception unit, and the signal processing unit will be described below.

### [2-2-1] Encoding Processing Unit

The encoding processing unit plays a role of performing the process of the compression-encoding method according to the present embodiment to compression-encode an image signal by each predetermined unit.

More specifically, the encoding processing unit transforms the frequency of the image signal by each predetermined unit, quantizes the frequency-transformed image signal, then allocates a code thereto, and thereby compression-encodes the image signal by the predetermined unit.

Here, the encoding processing unit transforms the frequency of the image signal using an arbitrary technique in which the frequency of the original signal can be expressed, for example, a wavelet transform, a discrete cosine transform, or the like.

In addition, the encoding processing unit increases, for example, distribution of quantization bits to low-order frequency components. Here, as the low-order frequency components, for example, a frequency component lower than a set threshold value is exemplified. The encoding processing unit specifies the set threshold value by, for example, reading data indicating the threshold value stored in a recording medium (not illustrated).

In addition, the encoding processing unit may reduce, for example, high-order frequency components so as to be inconspicuous. The encoding processing unit reduces the high-order frequency components by, for example, performing a filtering process using a filter such as a low-pass filter.

In addition, the encoding processing unit allocates a code using an arbitrary code, for example, a Huffman code, a run length code, an arithmetic code, or the like.

The encoding processing unit, for example, allocates a code having a fixed length to frequency components lower than the set threshold value and allocates a code having a variable length to frequency components other than the low frequency components.

Here, the frequency components lower than the threshold value are important frequency components that affect the quality of the medical captured image. By allocating the code having a fixed length to the frequency components lower than the threshold value as described above, even if an error occurs in a compression-encoding process, an interpolation process performed on an error pixel by the signal processing unit becomes easy, causing the error to be propagated only to pixels with the code having the fixed length. As the interpolation process performed on an error pixel by the signal processing unit, an arbitrary process in which the pixel value of the error pixel is interpolated, for example, a process of interpolating the pixel value of the error pixel using pixel values of the pixels surrounding the error pixel, or the like, is exemplified. That is, by allocating the code having the fixed length to the frequency components lower than the threshold value, even if the pixel is lost due to the error, it can be restored using data of the surrounding pixels. Therefore, by allocating the code having the fixed length to the frequency components lower than the threshold value, degradation of the quality of the decoded medical captured image can be prevented.

In addition, by allocating a code having a variable length to frequency components other than the low frequency components, a compression ratio of the image signal can be further increased.

Note that it is a matter of course that the encoding processing unit can allocate a code having a fixed length to all frequency components or a code having a variable length to all frequency components.

In addition, a process of changing a process in accordance with frequency components is not limited to the process of changing the code allocation method in accordance with frequency components.

For example, the encoding processing unit may not perform quantization on the frequency components lower than the set threshold value. Since the frequency components lower than the threshold value are important frequency components that affect the quality of the medical captured image as described above, the degradation of the quality of the decoded medical captured image can be prevented by not performing quantization on the frequency components lower than the threshold value.

The encoding processing unit compression-encodes, for example, the image signal by the predetermined unit as described above.

Here, the encoding processing unit compression-encodes the image signal at a set compression ratio. The encoding processing unit specifies the set compression ratio by, for example, reading data indicating the compression ratio stored in a recording medium (not illustrated).

As the set compression ratio, a pre-set fixed compression ratio is exemplified.

In addition, the set compression ratio may be, for example a compression ratio corresponding to a procedure set by selecting the procedure. In the medical observation system 1000, for example, the procedure is selected through a selection operation by a user using the medical observation system 1000, and data indicating the compression ratio corresponding to the selected procedure is stored in the recording medium (not illustrated). A process to record the data indicating the compression ratio corresponding to the selected procedure in the recording medium (not illustrated) may be performed by the medical observation device 100 (e.g., the control unit 114, etc.) or an external device such as a control device (not illustrated).

Note that a process of the encoding processing unit is not limited to the above-described process.

The encoding processing unit may add, for example, error correcting codes (ECC) to each predetermined unit. By adding the error correcting codes to increase redundancy, error resistance of compression-encoding can be further increased.

Adding the error correcting codes is disadvantageous for improving compression efficiency. However, further improving error resistance is beneficial when medical staff performing medical practice viewing the decoded medical captured image is taken into account.

In addition, the encoding processing unit may transform the frequency of the image signal after performing, for example, “pre-processing performed on the image signal such as color conversion, noise removal, band limitation, or image analysis,” or a “process of removing redundancy of the image signal by using motion compensation or the like.”

In addition, the encoding processing unit may change, for example, a way of compression-encoding in accordance with an operation state of a predetermined medical apparatus. As the predetermined medical apparatus according to the present embodiment, a treatment device, for example, an electric scalpel, forceps, or the like is exemplified.

In a case in which the predetermined medical apparatus is operated, there is a possibility of an electric field occurring due to the operation state of the predetermined medical apparatus affecting communication between the transmitter 110 and the receiver 112.

Thus, the encoding processing unit changes the way of compression-encoding on the basis of, for example, data indicating an operation state of the predetermined medical apparatus. The data indicating an operation state of the predetermined medical apparatus is acquired through communication with the predetermined medical apparatus (or a control device controlling the predetermined medical apparatus).

As examples in which the way of compression-encoding by the encoding processing unit is changed, for example, there are the following examples. Note that it is a matter of course that examples in which the way of compression-encoding by the encoding processing unit is changed are not limited to the following examples.

A compression ratio is changed to a compression ratio corresponding to an operation state of the predetermined medical apparatus.

The predetermined unit is changed to a unit corresponding to an operation state of the predetermined medical apparatus.

Whether to perform quantization on the frequency components lower than the set threshold value is switched in accordance with an operation state of the predetermined medical apparatus.

Whether to add error correcting codes is switched in accordance with an operation state of the predetermined medical apparatus.

A combination of two or more of the above-described examples.

The encoding processing unit specifies a way of compression-encoding in accordance with an operation state of the predetermined medical apparatus with reference to, for example, “a table (or a database) in which operation states of the predetermined medical apparatus are associated with data indicating the ways of compression-encoding” stored in a recording medium (not illustrated). In addition, the encoding processing unit may specify a way of compression-encoding in accordance with an operation state of the predetermined medical apparatus by, for example, performing an arithmetic operation of an arbitrary algorithm that can decide a way of compression-encoding in accordance with the operation state of the predetermined medical apparatus.

Then, the encoding processing unit compression-encodes the image signal by each predetermined unit using the specified way of compression-encoding in accordance with the operation state of the predetermined medical apparatus.

By changing the way of compression-encoding in accordance with the operation state of the predetermined medical apparatus as described above, for example, a switch to the method with strong error resistance is realized taking a transmission environment into consideration.

Note that a method of realizing the switch to the method with strong error resistance taking a transmission environment into consideration is not limited to the above-described example.

Since a medical apparatus to be used is often determined depending on a procedure to be performed, a medical apparatus to be used can be specified (anticipated) on the basis of the type of procedure to be used. Therefore, the encoding processing unit may change a way of compression-encoding in accordance with, for example, the type of procedure to be used.

In a case in which the procedure is selected through a selection operation of a user using the medical observation system 1000, for example, the encoding processing unit reads data indicating the way of compression-encoding corresponding to the selected procedure from a recording medium (not illustrated). In addition, the encoding processing unit may read the data indicating the way of compression-encoding corresponding to the procedure to be performed from the recording medium (not illustrated), for example, in cooperation with a device managing steps of medical practice such as surgery. Then, the encoding processing unit compression-encodes the image signal by each predetermined unit using the way of compression-encoding indicated by the data indicating the way of compression-encoding.

In addition, the encoding processing unit can also change the way of compression-encoding in accordance with, for example, both an operation state of the predetermined medical apparatus and the type of procedure to be used. For example, the encoding processing unit compression-encodes the image signal by each predetermined unit in the way of compression-encoding in accordance with the type of procedure to be performed and further change the way of compression-encoding in accordance with an operation state of the predetermined medical apparatus.

### [2-2-2] Transmission Unit

The transmission unit transmits the image signal compression-encoded by each predetermined unit. The transmission unit transmits the compression-encoded image signal in wired or wireless communication. Note that the transmission unit can also transmit an image signal that has not undergone compression-encoding.

### [2-2-3] Reception Unit

The reception unit receives the compression-encoded image signal transmitted from the transmission unit in wired or wireless communication. Note that the reception unit can also receive an image signal that has not undergone compression-encoding.

### [2-2-4] Signal Processing Unit

The signal processing unit processes the received compression-encoded image signal. Note that the signal processing unit can also process an image signal that has not undergone compression-encoding.

As a process on an image signal by the signal processing unit, one or two or more processes among various processes, for example, a decoding process (in a case in which the image signal has been compression-encoded), gamma correction, adjustment of white balance, enlargement or reduction of an image using the electronic zoom function, inter-pixel correction, and the like are exemplified.

The medical observation device 100 to which the compression-encoding method according to the present embodiment is applied has, for example, the encoding processing unit, the transmission unit, the reception unit, and the signal processing unit. Note that a configuration of the medical observation device 100 is not limited to the above-described example. In a case in which an external device functions as a reception side medical observation device, for example, the medical observation device 100 may not have the reception unit and the signal processing unit.

### [3] Example of Effects Exhibited by Using Medical Observation System According to Present Embodiment

Effects introduced in the following (1) to (5), for example, are exhibited in addition to the above-described effect by using the medical observation system according to the present embodiment. Note that it is a matter of course that effects exhibited by using the medical observation system according to the present embodiment are not limited to the following examples.

### (1) First Effect

A transmission capacity in communication between the transmitter and the receiver can be reduced by compression-encoding an image signal using the compression-encoding method according to the present embodiment.

### (2) Second Effect

By reducing the transmission capacity, power saving relating to transmission of an image signal can be achieved. In addition, by achieving power saving relating to transmission of an image signal, the amount of heat generated due to the transmission of the image signal is reduced accordingly, and thus the size of the medical observation device 100 (e.g., the size of the camera head 108 illustrated in FIG. 1) can be further miniaturized easily.

### (3) Third Effect

Since the number of lanes can be reduced by reducing the transmission capacity, the reduction contributes to reduction of cost for transmission of the image signal. To give an example, in a case in which the communication path T for transmission of image signals is a communication path in which optical fibers are used, it is possible to reduce the number of optical fibers and man-hours to adjust the optical axes of a plurality of optical fibers since the number of lanes is reduced.

### (4) Fourth Effect

By reducing the transmission capacity, for example, one or both of providing an imaging device with higher resolution in the medical observation device according to the present embodiment and providing a plurality of imaging devices in the medical observation device according to the present embodiment are possible. Then, as a result, medical captured images with higher quality can be provided to medical staff.

### (5) Fifth Effect

By reducing the transmission capacity, it is possible to perform wireless transmission of image signals more easily. In addition, by performing wireless transmission of image signals, for example, functionality of the camera head constituting an endoscope (an example of the medical observation device according to the present embodiment) can be improved.

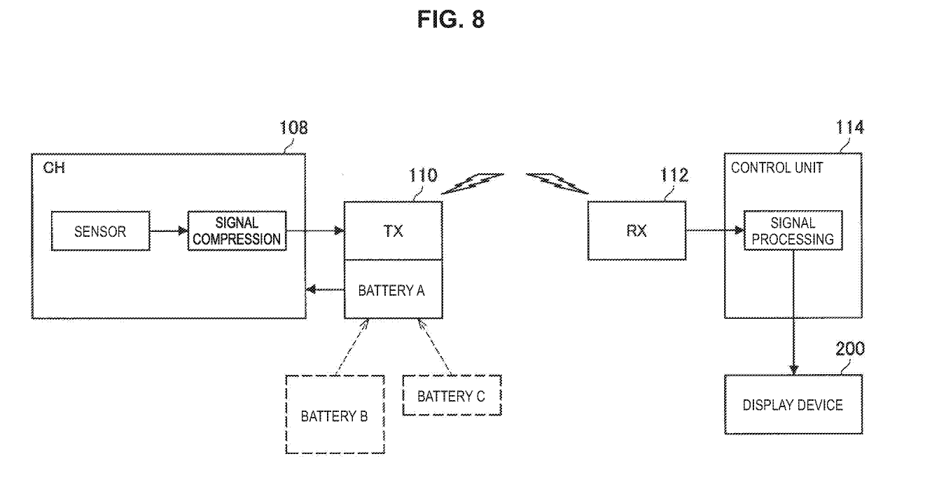

FIG. 8 is an explanatory diagram for describing an example of an effect exhibited by using the medical observation system according to the present embodiment. FIG. 8 illustrates a part of a configuration of the medical observation device 100 in a case in which the transmitter 110 and the receiver 112 included in the medical observation device 100 illustrated in FIG. 1 perform wireless communication.

The medical observation device 100 may have a replaceable battery that causes the camera head 108 and the transmitter 110 (an example of a constituent element of the transmission side medical observation device) to operate, for example, as illustrated in FIG. 8.

The medical observation device 100 can compression-encode an image signal in, for example, a way of compression-encoding in accordance with the type of procedure to be performed as described above. Thus, with the configuration in which the battery can be replaced as illustrated in FIG. 8, the camera head 108 or the transmitter 110 can be caused to operate with a battery in accordance with a type of a procedure to be performed (e.g., a battery of which power consumption at a compression ratio in accordance with the procedure is considered). Therefore, by having such a replaceable battery, for example, the weight of the camera head 108 side constituting the medical observation device 100 used in medical practice that is completed within a relatively short period of time can be further lightened. In addition, further lightening of the weight of the camera head 108 side leads to a reduction of a load imposed on medical staff using the medical observation device 100.

### (Program According to Present Embodiment)

By executing a program for causing a computer to function as the medical observation device according to the present embodiment (e.g., a program for causing the computer to function as the encoding processing unit, i.e., a program that can execute the process of the compression-encoding method according to the present embodiment) by a processor or the like in the computer, an image signal indicating a medical captured image can be compression-encoded while the influence of an error caused by the compression-encoding is further reduced.

In addition, by executing the program for causing the computer to function as the medical observation device according to the present embodiment by the processor or the like in the computer, the above-described effects to be exhibited by performing the process of the compression-encoding method according to the present embodiment can be exhibited.

It should be understood by those skilled in the art that various modifications, combinations, sub-combinations and alterations may occur depending on design requirements and other factors insofar as they are within the scope of the appended claims or the equivalents thereof.

Although it has been described above that, for example, the program (computer program) for causing the computer to function as the medical observation device according to the present embodiment is provided, the present embodiment can also provide a recording medium in which the program is stored therealong.

The above-described configuration is an example of the present embodiment, and of course belongs to the technical scope of the present disclosure.

Further, the effects described in this specification are merely illustrative or exemplified effects, and are not limitative. That is, with or in the place of the above effects, the technology according to the present disclosure may achieve other effects that are clear to those skilled in the art from the description of this specification.

Additionally, the present technology may also be configured as below.
(1) A medical observation device including:

an encoding processing unit configured to compression-encode an image signal indicating a medical captured image by predetermined unit that is smaller than the medical captured image, the medical captured image being obtained by capturing an observation target by an imaging device; and

a transmission unit configured to transmit the image signal compression-encoded by the predetermined unit.
(2) The medical observation device according to (1), in which the encoding processing unit transforms a frequency of the image signal, quantizes the frequency-transformed image signal, and allocates a code to the image signal, and thereby compression-encodes the image signal by the predetermined unit.
(3) The medical observation device according to (2), in which the encoding processing unit allocates a code having a fixed length to a frequency component lower than a set threshold value and allocates a code having a variable length to a frequency component other than the low frequency component.
(4) The medical observation device according to (2) or (3), in which the encoding processing unit does not quantize a frequency component lower than a set threshold value.
(5) The medical observation device according to any one of (1) to (4), in which the encoding processing unit adds an error correcting code by the predetermined unit.
(6) The medical observation device according to any one of (1) to (5), in which the encoding processing unit compression-encodes the image signal at a set compression ratio.
(7) The medical observation device according to (6), in which the compression ratio is a compression ratio corresponding to a procedure set by selecting the procedure.
(8) The medical observation device according to (6), in which the compression ratio is a pre-set fixed compression ratio.
(9) The medical observation device according to any one of (1) to (8), in which the predetermined unit is a unit of a plurality of lines of the medical captured image.
(10) The medical observation device according to any one of (1) to (9), in which the encoding processing unit changes a way of compression-encoding in accordance with one or both of an operation state of a predetermined medical apparatus and a type of a procedure to be performed.
(11) The medical observation device according to any one of (1) to (10), in which the transmission unit transmits the compression-encoded image signal in wired communication.
(12) The medical observation device according to any one of (1) to (10), in which the transmission unit transmits the compression-encoded image signal in wireless communication.
(13) The medical observation device according to any one of (1) to (12), including:

the imaging device configured to be inserted into an inside of a body of a patient and image the inside of the body.
(14) The medical observation device according to any one of (1) to (12), including:

an arm including a plurality of links connected to each other by joints; and

the imaging device supported by the arm.
(15) A medical observation system including:

a transmission side medical observation device including
an encoding processing unit configured to compression-encode an image signal indicating a medical captured image by predetermined unit that is smaller than the medical captured image, the medical captured image being obtained by capturing an observation target by an imaging device, and
a transmission unit configured to transmit the image signal compression-encoded by the predetermined unit; and

a reception side medical observation device including
a reception unit configured to receive the compression-encoded image signal transmitted from the transmission side medical observation device, and
a signal processing unit configured to process the received compression-encoded image signal.

What is claimed is:

1. A medical observation device comprising:
an encoding processing unit configured to compression-encode an image signal indicating a medical captured image by predetermined unit that is smaller than the medical captured image, the medical captured image being obtained by capturing an observation target by an imaging device; and
a transmission unit configured to transmit the image signal compression-encoded by the predetermined unit.

2. The medical observation device according to claim 1, wherein the encoding processing unit transforms a frequency of the image signal, quantizes the frequency-transformed image signal, and allocates a code to the image signal, and thereby compression-encodes the image signal by the predetermined unit.

3. The medical observation device according to claim 2, wherein the encoding processing unit allocates a code having a fixed length to a frequency component lower than a set threshold value and allocates a code having a variable length to a frequency component other than the low frequency component.

4. The medical observation device according to claim 2, wherein the encoding processing unit does not quantize a frequency component lower than a set threshold value.

5. The medical observation device according to claim 1, wherein the encoding processing unit adds an error correcting code by the predetermined unit.

6. The medical observation device according to claim 1, wherein the encoding processing unit compression-encodes the image signal at a set compression ratio.

7. The medical observation device according to claim 6, wherein the compression ratio is a compression ratio corresponding to a procedure set by selecting the procedure.

8. The medical observation device according to claim 6, wherein the compression ratio is a pre-set fixed compression ratio.

9. The medical observation device according to claim 1, wherein the predetermined unit is a unit of a plurality of lines of the medical captured image.

10. The medical observation device according to claim 1, wherein the encoding processing unit changes a way of compression-encoding in accordance with one or both of an operation state of a predetermined medical apparatus and a type of a procedure to be performed.

11. The medical observation device according to claim 1, wherein the transmission unit transmits the compression-encoded image signal in wired communication.

12. The medical observation device according to claim 1, wherein the transmission unit transmits the compression-encoded image signal in wireless communication.

13. The medical observation device according to claim 1, comprising:
the imaging device configured to be inserted into an inside of a body of a patient and image the inside of the body.

14. The medical observation device according to claim 1, comprising:
an arm including a plurality of links connected to each other by joints; and
the imaging device supported by the arm.

15. A medical observation system comprising:
a transmission side medical observation device including
an encoding processing unit configured to compression-encode an image signal indicating a medical captured image by predetermined unit that is smaller than the medical captured image, the medical captured image being obtained by capturing an observation target by an imaging device, and
a transmission unit configured to transmit the image signal compression-encoded by the predetermined unit; and
a reception side medical observation device including
a reception unit configured to receive the compression-encoded image signal transmitted from the transmission side medical observation device, and
a signal processing unit configured to process the received compression-encoded image signal.
